(19) United States
(12) Patent Application Publication
(10) Pub. No.: US 2021/0393940 A1
(43) Pub. Date: Dec. 23, 2021

(54) Stopcock

(71) Applicant: Youn Joo Kim, Busan (KR)

(72) Inventor: Youn Joo Kim, Busan (KR)

(21) Appl. No.: 17/287,261

(22) PCT Filed: Oct. 24, 2019
(86) PCT No.: PCT/KR2019/014093

§ 371 Date: Apr. 21, 2021

(30) Foreign Application Priority Data
Oct. 24, 2018 (KR) 10-2018-0127468
Oct. 24, 2019 (KR) 10-2019-0132832

(51) Int. Cl. A61M 39/24; A61M 39/02; A61M 39/20

(52) U.S. Cl. CPC A61M 39/24; A61M 2039/229; A61M 39/20; A61M 39/0208

(57) ABSTRACT

Proposed is a stopcock that enables natural injection of a drug and prevents backflow of a drug injected in another direction on a body. The stopcock includes: a body formed in a passage shape connecting a drug solution injection pipe and a supply pipe to each other; and a backflow prevention unit having a first end coupled to a side of the body between the drug solution injection pipe and the supply pipe and a second end extending toward an inside of the body to isolate the drug solution injection pipe and the supply pipe from each other, in which the second end of the backflow prevention unit bends toward the inside of the body and guides movement of a drug when the drug is injected through the drug solution injection pipe, and elastically moves to an initial position when injection of the drug is stopped.

### CROSS-REFERENCE TO RELATED APPLICATION

This Application is a Section 371 National Stage Application of International Application No. PCT/KR2019/014093, filed Oct. 24, 2019, the contents of which is hereby incorporated by reference in its entirety.

### TECHNICAL FIELD

The present disclosure relates to a stopcock and, more particularly, to a stopcock connected to transfusion tubes and controlling movement of drugs.

### BACKGROUND ART

Unless stated otherwise in this specification, the contents described in this section are not the related art about the claims of this application and not ail of the contents included in this section are regarded as the related art.

Drugs including a saline solution, a liquid drug, and a drug solution are supplied to the body of a patient through transfusion tubes and a stopcock is connected to the transfusion tubes, thereby controlling movement of various drugs.

When movement of drugs is controlled by an existing 3-way stopcock, the passage for injecting a drug is opened or the passage for stopping injection of a drug is closed by rotating a valve of the stopcock.

However, there is a large possibility, that a drug flows into the valve and is contaminated when the valve is rotated and there is a defect. That the stopcock itself is contaminated and the lifespan thereof decreases due to contamination of the valve, so there is a need. For a technology of preventing contamination of the valve.

In relation to this, a medical stopcock and valve unit has been disclosed in Korean Patent No. 0901729 and a functional multi-directional medical stopcock has been disclosed in Korean Patent No. 10-1404488.

However, these documents do not propose a technology of minimizing or preventing contamination by operation of a value.

### DISCLOSURE

### Technical Problem

An objective of the present disclosure is to provide a stopcock that enables natural injection of a drug and prevents backflow a drug injected in another direction on a body.

The present disclosure is not limited to the objectives and it is apparent that other objectives may be derived from the following description.

### Technical Solution

According to an embodiment of the present. Disclosure, a stopcock includes: a body formed in a passage shape connecting a drug solution injection pipe and a supply pipe to each other; and a backflow prevention unit having a first end coupled to a side of the body between the drug solution injection pipe and the supply pipe and a second end extending toward an inside of the body to isolate the drug solution injection pipe and the supply pipe from each other, in which the second end of the backflow prevention unit bends toward the inside of the body and guides movement of a drug when the drug is injected through the drug solution injection pipe, and elastically moves to an initial position when injection of the drug is stopped.

The first end of the drug solution injection pipe may be formed in a cylinder shape, the second end of the drug solution injection pipe may be formed by extending in a plate shape from a portion of an edge of the first end in close contact with an inner surface of the body, and a groove recessed toward the drug solution injection pipe may be formed at the second end.

The body may further include a drug injection pipe coupled to an outer side of the body at a position spaced apart from the backflow prevention unit between the drug solution injection pipe and the supply pipe, and having a second end extending outward in a tube shape and connecting the inside of the body to the outside.

The stopcock may further include a drug injection port having a first end formed in a tube shape and detachably coupled to the second end of the drug injection pipe, and a second end extending away from the body to seal the drug injection pipe.

The drug injection port may include: a housing having a first end formed in a tube shape and surrounding the drug injection pipe and a second end extending and tapering away from the body; and a cap having a first end inserted in the second end of the housing, a second end expanding and extending toward the body from an edge of the first end in close contact with an inner surface of the drug injection pipe, and a hole formed through a side thereof.

The backflow prevention unit may have a first end having a ring-shaped protrusion surrounding an outer side thereof, and inserted in a side of the body, and a second end extending into the body in close contact with an inner surface of the body from a portion of an edge of the first end.

The stopcock may further include a shock absorber formed inside another side of the body to be spaced apart from the backflow prevention unit with a predetermined space formed in the body therebetween.

The stopcock may further include a connection cap having a first end coupled to be able to rotate with the supply pipe, a second end expanding and extending from the first end, and a thread formed inside the second end to be engaged with an outer side of the drug solution injection pipe.

### Advantageous Effects

According to an embodiment of the present disclosure, the stopcock has an advantage that a drug can be naturally injected without specific operation and another drug inserted in another direction is prevented from moving to a drug injection port formed at another position.

The stopcock has an advantage that the backflow prevention unit is detachably coupled, is formed in a shape that can be easily formed, and is made of a polymeric material, so the manufacturing cost is reduced and maintenance is easy.

The stopcock has an advantage that since an elastic shock absorber is disposed therein, a needle inserted through a side is prevented from being damaged by hitting against the inside of the stopcock and the deformation ability of the backflow prevention unit is controlled in accordance with a rotation angle.

The effects of the present disclosure are naturally achieved by the configuration described herein regardless of whether the inventor(s) recognizes the effects, so the effects are only some effects according to the description and should not be admitted as all effects that the inventor (s) knows or that actually exist.

Further, effects of the present disclosure should be additionally checked through the entire specification, and if an effect is an effect that can be admitted by those skilled in the art on the basis of the specification, the effect should be considered as an effect described herein even though it is not clearly stated herein.

### DESCRIPTION OF DRAWINGS

FIGS. 1 and 2 are perspective views showing a stopcock according to an embodiment of the present disclosure;

FIG. 3 is an exploded perspective view of the stopcock shown in FIG. 1;

FIG. 4 is a cross-sectional view of the stopcock shown in FIG. 2 taken along line I-I′;

FIG. 5 is a cross-sectional view of the stopcock shown in FIG. 2 taken along line II-II′;

FIG. 6 is a perspective view showing a stopcock according to another embodiment of the present disclosure

FIGS. 7 and 8 are perspective views showing a stopcock according to another embodiment of the present disclosure;

FIG. 9 is a cross-sectional view of the stopcock shown in FIG. 7 taken along line III-III′;

FIG. 10 is a perspective view showing a backflow prevention unit of FIG. 7 in different directions;

FIG. 11 is a cross-sectional view showing a stopcock according to another embodiment of the present disclosure;

FIG. 12 is a perspective view showing examples of another state of the stopcocks shown in FIGS. 1 and 11;

FIG. 13 is a perspective view showing a stopcock according to another embodiment of the present disclosure; and

FIG. 14 is a cross-sectional view of the stopcock shown in FIG. 13 taken along line IV-IV′;

### MODE FOR INVENTION

The configuration, operation, and operation effects of stopcocks according to exemplary embodiments of are described hereafter with reference to the accompanying drawings. For reference, the components are not shown or are schematically shown in the drawings for the convenience and clearness of the following description and the sizes of the components do not reflect the actual sizes. Further, the same reference numerals indicate the same components throughout the specification and the reference numerals for the same components are omitted in each drawing.

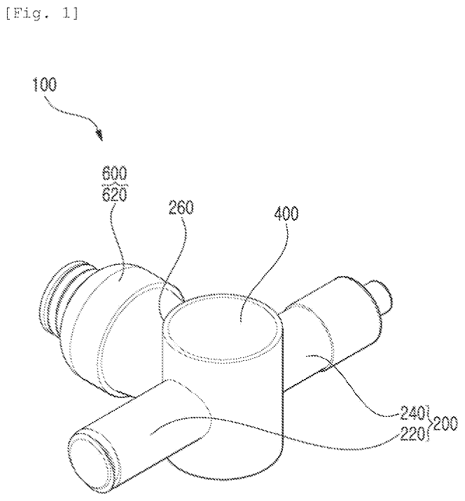

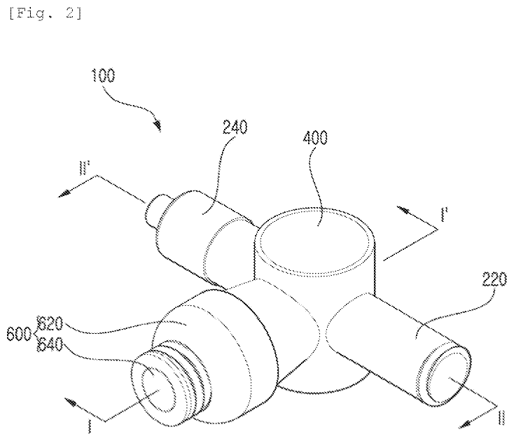

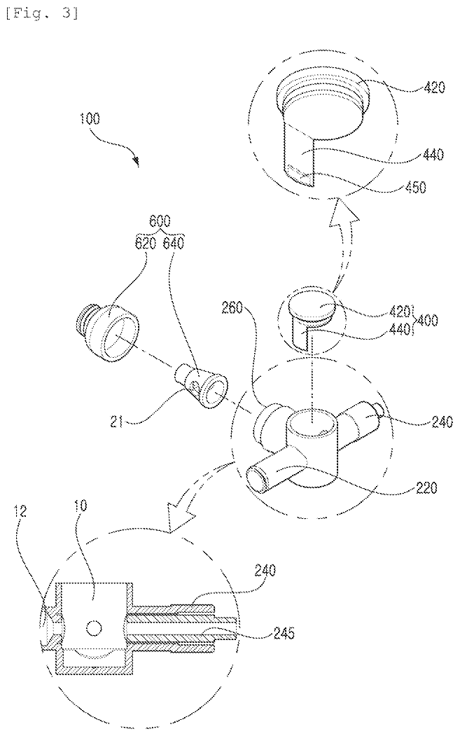

FIGS. 1 and 2 are perspective views showing a stopcock according to an embodiment of the present disclosure. FIG. 3 is an exploded perspective view of the stopcock shown in FIG. 1.

As shown in FIGS. 1 to 3, a stopcock 100 includes a body 200, a backflow prevention unit 400, and a drug injection port 600.

The stopcock 100 controls movement of drugs including a saline solution, a liquid drug, and a drug solution that are prescribed to a patient, can simultaneously give various drugs, and effectively prevents backflow depending on injection of various drugs without specific operation.

The body 200 includes a drug solution injection pipe 220, a supply pipe 240, and a drug injection pipe 260.

The body 200 is formed in a cylindrical shape with an internal space and has a passage 10 connecting the drug solution injection pipe 220 and the supply pipe 240 coupled to the outer surface of the body 200 so each other.

A first end of the drug solution injection pipe 220 is coupled to the body 200 while surrounding an injection port connected to the passage through the body 200 at a position corresponding to the front of the body 200, and a second end thereof extends away from the body 200 in a pipe shape.

A first end of the supply pipe 240 is coupled to the body 200 while surrounding a rear injection port connected to the passage through the body 200 at a position corresponding to the rear of the body 200, and a second end thereof extends away from the body 200 in a pipe shape.

A tube 245 cylindrically surrounding the internal space of the supply pipe 240 is disposed in the supply pipe 240, so a drug flowing in the passage 10 can be discharged outside through the internal space of the supply pipe 240.

A first end of the drug injection pipe 260 is coupled to the outer surface of the body 200 at a distance from the backflow prevention unit 400 between the drug solution injection pipe 220 and the supply pipe 240, and a second end thereof extends outward from the body 200 to connect the passage 10 in the body 200 to the outside.

The backflow prevention unit 400 includes a cap 420 and a blocking diaphragm 440.

A first end of the backflow prevention unit 400 is coupled to a side of the body 200 between the drug solution injection pipe 220 and the supply pipe 240, and a second end thereof extends into the body 200 and passes through the body 200 to isolate the drug solution injection pipe 220 and the body 200 from each other.

A first end of the cap 420 is formed in a circular plate shape and a second end thereof extends a predetermined distance downward from a circular center portion excluding the edge of the first end. The cap 420 is inserted in the upper portion of the body 200, thereby isolating the passage 10 from the outside.

A user can inject a desired drug into the passage 10 in the body 200 by inserting the needle of a syringe through the cap 420 with the backflow prevention unit 400 coupled to the body 200.

An end of the blocking diaphragm. 440 is in contact with a portion of the second end of the cap 420 at a position corresponding to the front of the cap 420 and a second end thereof extends downward while maintaining a semicircular shape that is convex forward.

The rear surface of the blocking diaphragm 440 is a flat surface and the rear surface of the second end of the blocking diaphragm 440 is partially recessed forward a predetermined distance, whereby a groove 450 is formed on the rear surface of the second end of the blocking diaphragm 440.

The blocking diaphragm 440 is in close contact with or spaced a predetermined distance apart from the inner surface of the body 200, which corresponds to the position to which the first end of the drug solution injection pipe 220 is connected, in the passage 10, thereby isolating a passage 12 in the drug solution injection pipe 220 and the passage 10 in the body 200 from each other.

The second end of the blocking diaphragm 440 is in close contact with a step formed by a portion of the inner side of the body 200 which cylindrically extends toward the inside of the passage 10. The second end of the blocking diaphragm 440 is in close contact with the step in a non-operation state, but is separated from the step by pressure of a drug moving from the passage 12 to the passage 10.

The blocking diaphragm 440 is made of an elastic polymeric or natural material. When a drug is sent to the passage 10 through the drug solution injection pipe 220 with the front surface of the blocking diaphragm 440 in close contact with the inner surface of the body 200, the blocking diaphragm 440 is bent toward the passage 10 by the pressure of the drug, whereby a space through which the drug can move is formed between the passage 10 and the passage 12.

When the drug stops being supplied through the drug solution injection pipe 220, the blocking diaphragm 140 comes in close with the inner surface of the body 200 by elastically returning to the initial position and the passage 10 and the passage 12 are isolated from each other.

A drug flowing into the passage 10 through the supply pipe 240 or the drug injection port 600 is prevented from flowing backward to the passage 12 by the blocking diaphragm 440 and the second end of the blocking diaphragm 440 corresponding to the inside of the groove 450.

A drug flowing into the groove 450 formed at the second end of the blocking diaphragm 440 presses the blocking diaphragm 440 while flowing into the groove 450, thereby improving the sealing ability between the blocking diaphragm 440 and the inner surface of the body 200.

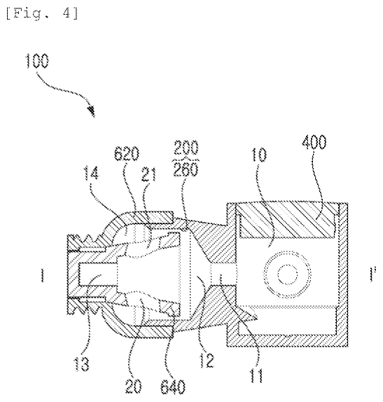

FIG. 4 is a cross-sectional view of the stopcock shown in FIG. 2 taken along line I-I′.

As shown in FIGS. 1 to 4, the drug injection port 600 includes a housing 620 and a cap 640.

A first end of the drug injection port 600 is formed in a tube shape and is detachably coupled to the second end of the drug injection pipe 260, and a second end thereof extends away from the body 200 to seal the drug injection pipe 260.

A first end of the housing 620 is formed in a tube shape and is in close contact with she drug injection pipe 620 while surrounding a portion of the second end of the drug injection pipe 260, and a second end thereof curves and extends to be narrowed away from the body 200 and keeps extending in a cylindrical shape.

A thread is formed out the outer surface of the second end of the housing 620 to be firmly connected to a vial through a tube, and a passage 14 going through the housing 620 is formed inside the second end.

A first end of the cap 640 is cylindrically shape and is inserted inside the second end of the housing 620 in the passage 14, and a second end thereof expands toward the second end of the housing 620 from the edge of the first end, extends at an angle, and is positioned in the drug injection pipe 260.

Holes 20 and 21 facing each other and connecting the passage 13 and the passage 14 to each other are formed through both sides of the cap 640 and the first end of the cap 640 is positioned on the same horizontal line as a connection pipe 11 passing through the drug injection pipe 260 between the passage 10 and the passage 12.

Accordingly, a drug injected into the passage 10 through the first end of the cap 640, the passage 13, and the connection pipe 11 moves into the supply pipe 240 through the passage 10. A drug flowing backward to the passages 12, 13, and 14 through the connection pipe 11 moves to the space between the first end of the housing 620 and the second end of the cap 640 through the hole 20, whereby the drug is prevented from flowing backward to the outside through the cap 640.

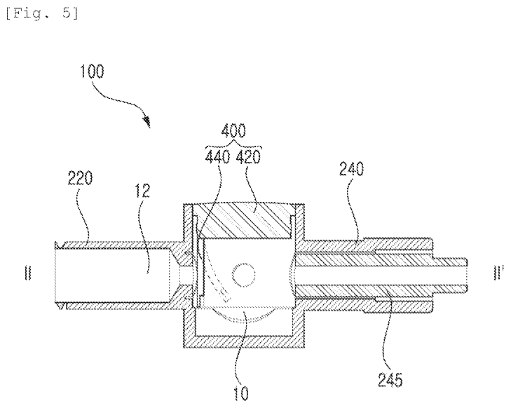

FIG. 5 is a cross-sectional view of the stopcock shown in FIG. 2 taken along line II-II′.

As shown in FIGS. 1 to 5, a first drug moving toward the passage 10 of the body 200 from the passage 12 with the cap 420 and the blocking diaphragm 440 inserted in the passage 10 and coupled to the body 220 moves into the body 200 while pushing the second end of the blocking diaphragm 440 toward the passage 10.

When a second drug flows into the passage 10 through the drug injection port 600 while the first drug flows into passage after passing through the blocking diaphragm. 440, the blocking diaphragm 440 comes in close contact with the inner surface of the body 200 or covers the inner surface of the body 200 by the injection pressure of the second drug and isolates the passage 10 and the passage 12 from each other, thereby preventing backflow of the second drug.

When a third drug flows into the passage 10 through an injector injected through the cap 420 while the first drug flows into the passage 10 after passing through the blocking diaphragm 440, the blocking diaphragm 440 comes in close contact with the inner surface of the body 200 or covers the passage 12, and the passage 10 and the passage 12 are isolated by the elastic restoring force of the blocking diaphragm 440 or the injection pressure of the third drug.

Accordingly, the stopcock. 100 effectively naturally prevents other drugs from flowing backward into the drug solution injection pipe 220 that are injected through other portions of the body 200 without specific operation while a drug is continuously injected into the body 200 through the drug solution injection pipe 220.

Further, the stopcock 100 has an advantage that it is possible to inject different drugs into the passage 10 in the body 200 in three directions through the drug solution injection pipe 220, the backflow prevention unit 400, and the drug injection port 600 while naturally preventing a drug from flowing backward to the drug solution injection pipe 220.

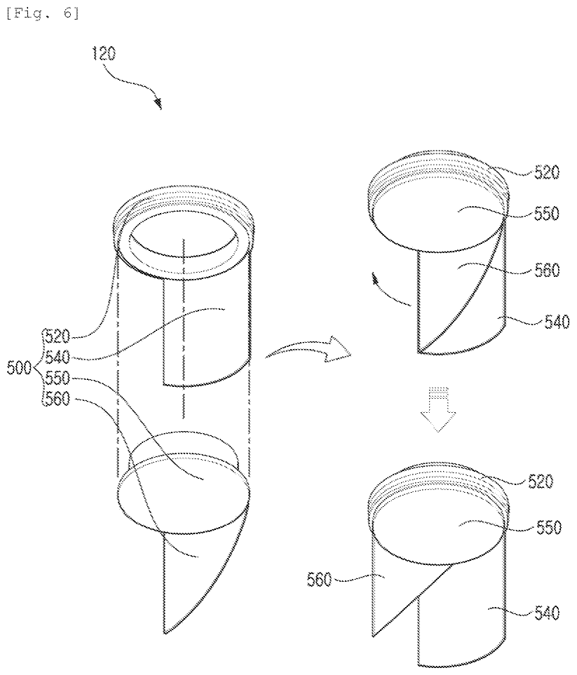

FIG. 6 is a perspective view showing a stopcock according to another embodiment of the present disclosure.

Since a stopcock 120 according to this embodiment is substantially the same as the stopcock 100 show in FIGS. 1 to 5 except for a backflow prevention unit 500, the same reference numerals and terms are used and repeated description is omitted.

As shown in FIG. 6, the stopcock 120 includes a backflow prevention unit 500.

According to the stopcock 120, it is possible to control the inflow speed or the flow rate of a drug flowing into the passage 10 through the backflow prevention unit 500 from the drug solution injection pipe 220 by partially rotating the backflow prevention unit 500.

The backflow prevention unit 500 includes a cap 520, a blocking diaphragm 540, a valve 550, and an assistant diaphragm 560.

A first end of the backflow prevention unit 500 is coupled to a side of the body 200 between the drug solution injection pipe 220 and the supply pipe 240, and a second end thereof extends into the body 200 to isolate the drug solution injection pipe 220 and the body 200 from each other.

A part of the backflow prevention unit 500 is rotatable coupled to the center of the first end of the backflow prevention unit 500, so it is possible to control the inflow speed or flow rate of a drug moving to the passage 10 from the drug solution injection pipe 220 by rotating the part.

A first end of the cap 520 is inserted in the upper end of the body 200 in close contact with the inner surface of the body 200 and a second end thereof extends downward along the inner surface of the body 200. A groove having a semicircular cross-section, recessed toward the inside of the cap 520, and surrounding the cap 520 is formed on the outer surface of the second end.

A ring-shaped protrusion formed at the upper end of the body 200 is inserted in the groove formed on the outer surface of the cap 520, whereby the coupling force between the cap 520 and the body 200 is improved and a drug flowing in the passage 10 is prevented from leaking outside through the cap 520.

A first end of the blocking diaphragm 540 is coupled to the edge of the front of the cap 520 and a second end thereof is in close contact with the body 200 and extends downward in a plate shape, thereby isolating the passage 10 and the passage 12 from each other.

Both edges of the blocking diaphragm 540 bends rearward such that the center portion of the front surface and both sides are in contact with the inner surface of the body 200 at the joint between the passage 10 and the passage 12, thereby isolating the passage 10 and the passage 12 from each other.

A first end of the valve 550 is cylindrically formed and coupled to be able co rotate along the inner surface of the cap 520 and a second end thereof expands outward along the bottom of the second end of the cap 520 from the lower portion of the second end of the cap 520 in close contact with the bottom of the cap 520.

A first end of the assistant diaphragm 560 is coupled to a portion of the edge of the bottom of the second end of the valve 550 and a second end thereof extends in a triangular plate shape having a side extending downward to be relatively long in close contact with the rear surface of the blocking diaphragm 540.

Both sides of the assistant diaphragm 50 are partially bent rearward such that the center portion of the front surface and both sides are in close contact with the rear surface of the blocking diaphragm 540, thereby controlling the degree of deformation of the blocking diaphragm 540 by the pressure of a drug applied to the front surface of the blocking diaphragm 540.

A user can align both edges of the assistant diaphragm 560 with both edges of the blocking diaphragm 540 by rotating the valve 550 such that the front surface of the assistant diaphragm 560 comes in close contact with the rear surface of the blocking diaphragm 540.

In this case, when the pressure of a drug moving from the passage 12 to the passage 10 is constant, the area of the joint where the passage 12 and the passage 10 can be connected to each other by bending of the blocking diaphragm 540 is relatively small, so the speed or the amount of the drug flowing into the passage 10 decreases.

On the other hand, when the area of the assistant diaphragm 560 coming in contact with the blocking diaphragm 510 decreases by moving the side of the assistant diaphragm. 560, which extends downward to be relatively long, toward a side of the assistant diaphragm 560 by rotating the valve 550, the bendable area of the blocking diaphragm. 540 increases, so the inflow speed or the flow rate of a drug flowing into the passage 10 increases.

Accordingly, according to the stopcock 120, the bendable area of the blocking diaphragm 540 is controlled in accordance with the rotation direction of the valve 550, whereby it is possible to conveniently control the inflow speed or the flow rate of a drug moving into the body 200 through the drug solution injection pipe 220.

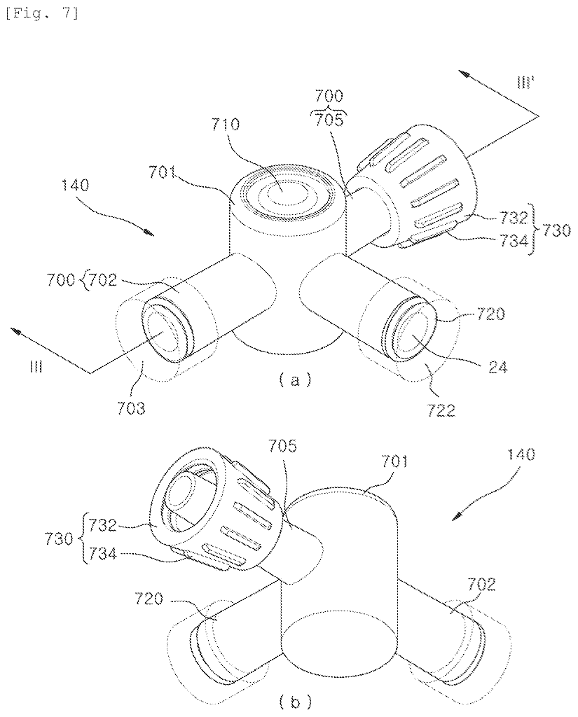

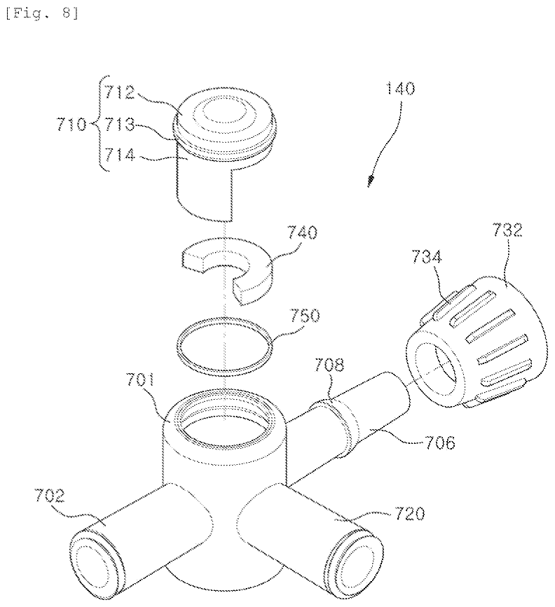

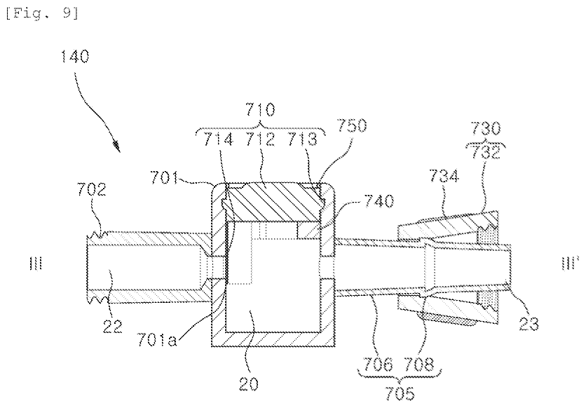

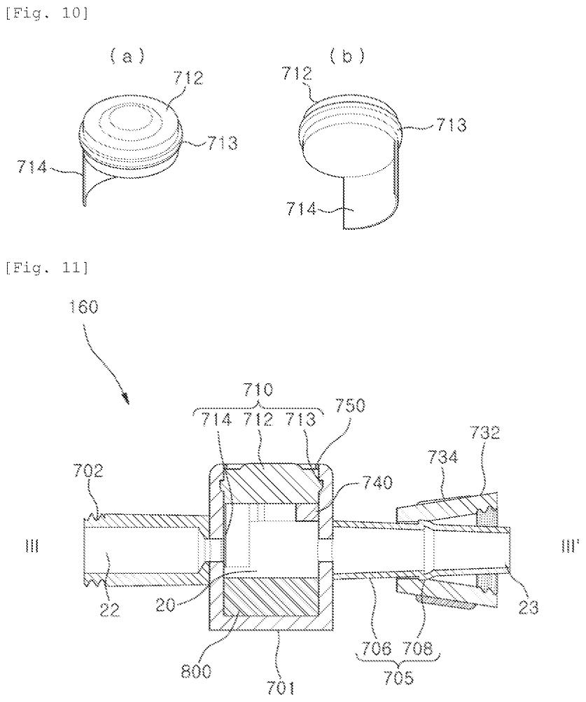

FIGS. 7 and 8 are perspective views showing a stopcock according to another embodiment of the present disclosure. FIG. 9 is a cross-sectional view of the stopcock shown in FIG. 1 taken along line FIG. 10 is a perspective view showing a backflow prevention unit of FIG. 7 in different directions.

As shown in FIGS. 7 to 10, a stopcock 140 includes a body 100, a backflow prevention unit 710, a drug injection port 720, a connection cap 730, a stopper 740, and a ring 750.

The stopcock 140 can prevent backflow of a drug flowing therein and can enable various drugs to be injected at various positions. A plurality of stopcocks 140 can be connected.

The body 700 includes a center part 710, a drug solution injection pipe 702, a cap 703, and a supply pipe 705.

The body 700 is elongated forward or rearward, has passages elongated in the extension direction therein, and has a center portion having a cylindrical shape partially extending perpendicular to the extension direction.

The center part 701 is cylindrically elongated upward or downward such that the space therein is open upward, and connects the drug solution injection pipe 702 and the supply pipe 705, which are formed at the front and rear, respectively, to each other.

A first end of the drug solution injection pipe 702 is cylindrically formed and connected to the center part 701 at a position corresponding to the front of the center part 701 and a second end thereof extends a predetermined distance forward while maintaining the cylindrical shape.

The cap 703 is formed in a cylindrical shape having a cylindrical internal space being open rearward, and a thread for engagement with the thread formed on the outer surface of the second end of the drug solution injection pipe 702 is formed on the inner surface of the cap 703.

Accordingly, a user can close the drug solution injection pipe 702 by thread-fastening the cap 703 to the second end of the drug solution injection pipe 702, and can inject a drug through the drug solution injection pipe 702 after separating the cap 103 when the drug solution injection pipe 702 is used.

A first end of the supply pipe cylindrically formed and connected to the center part 701 at a position corresponding to the rear of the center part 701 and a second end thereof extends a predetermined distance rearward while maintaining the cylindrical shape such that the inner diameter and the outer diameter decrease.

The outer diameter of the second end of the supply pipe 705 is relatively smaller than or the same as the outer diameter of the second end of the drug solution injection pipe 702. When a plurality of stopcocks 140 is disposed front and rear and the second ends of the supply pipes 705 are inserted in the second ends of the drug solution injection pipe 702, the stopcocks 140 are connected to each other.

The drug solution injection pipe 102 and the supply pipe 705 are connected to the center part 701 and the internal spaces of the drug solution injection pipe 702 and the supply pipe 702 are connected to the internal space of the center part 701, whereby passages are formed.

Referring to FIGS. 8 and 9, the supply pipe 705 has a supply pipe 706 and a guide 708.

A first end of the supply body 706 cylindrically formed and is connected to the rear of the center part 701 and a second end thereof extends a predetermined distance, is tapered while maintaining the cylindrical shape, and surrounds the passage 23.

The guide 708 protrudes outward in a ring shape between a first end and a second end of the supply body 706 and has a cross-sectional shape of which the front has a straight line shape vertically extending and the rear is curved rearward and extends outward from the supply body 706, thereby entirely having a sail shape.

The backflow prevention unit 710 has a cap 712, a protrusion 713, and a blocking diaphragm 714.

A first end of the backflow prevention unit 710 has a ring-shaped protrusion surrounding the outer side and is inserted in the upper portion of the center part 101 such that the protrusion is inserted in the inner surface of the center part 701, and a second end thereof extends a predetermined distance downward from a portion of the edge of the first end in a plate shape having an arc cross-section.

The cap 712 is cylindrically formed and has a ring-shaped protrusion 713 formed at the center of the outer surface and surrounding the outer side of an end, and a portion of the top of the cap 721 protrudes upward in a circular plate shape.

The cap 712 is made of a polymeric or natural material for easy penetration of a needle, and a user can insert the needle of a syringe into the center part 701 through the cap 712 and can inject the needle in the syringe into the center part 701.

The protrusion 713 is formed in a ring shape surrounding the center of the outer side of the cap 712, is integrated with the cap 712, and has a cross-section of which the upper end is formed in a rectangular parallelepiped shape and the lower end of the cross-section is formed such that the outer edge is tapered in a curvilinear shape.

Accordingly, referring to FIG. 9, there is an advantage that a user can easily insert the backflow prevention unit 710 into the center part 701 by the curved surface of the lower end edge. Further, when the protrusion 713 is inserted in the inner surface of the upper end of the center part 701, the passage 20 in the center part 20 is closed, so separation or deviation of the backflow prevention unit 710 is prevented.

A first end of the blocking diaphragm. 714 is formed in an arc shape that has a cross-section protruding forward and is coupled to a portion of the lower front edge of the cap 712, and a second end thereof extends a predetermined distance downward from the first end while maintaining the arc cross-section.

Referring to FIG. 9, the passage 22 in the drug solution injection pipe 702 connected to the front of the center part 701 extends rearward while relatively tapering and is connected the passage 20, and the second end of the blocking diaphragm 711 extends downward in close contact with the front inner surface 701a of the center part 701, thereby isolating the passages 20 and 22 from each other.

The blocking diaphragm. 714 is made of an elastic metal, polymeric, or natural material. When a drug moves from the passage 22 to the passage 20 through the drug solution injection pipe 702 with the front surface of the blocking diaphragm 714 in close contact with the inner surface 701a of the center part 701, the blocking diaphragm 114 is bent toward the passage 20 by the pressure of the drug and a space through which the drug can move is formed between the passages 20 and 22.

On the other hand, when the drug stops being supplied through the drug solution injection pipe 702, the blocking diaphragm. 711 elastically moves to the initial position and comes in close contact with the inner surface 701a, whereby the space between the passages 20 and 22 is removed.

Accordingly, the blocking diaphragm 714 naturally prevents another drug injected in any one of the backflow prevention unit 710, the drug injection port 720, or the supply pipe 705 from moving backward to the passage 22 in the drug solution injection pipe 702 through the passage 20.

The drug injection port 720 includes a cap 722.

A first end of the drug injection port 720 is cylindrically formed and connected to the center part. 701 at a position corresponding to a side of the center part 701 and a second end thereof extends a predetermined distance toward the side of the center part 701 from the first end.

The cap 722 is formed in a cylindrical shape having a cylindrical internal space being open to another side of the center part 701, and a thread for engagement with the thread formed on the outer surface of the second end of the drug injection port 720 is formed on the inner surface of the cap 722.

Accordingly, a user can close the drug injection port 720 by thread-fastening the cap 722 to the second end of the drug injection port 720, and can inject a drug into the center part 710 through the drug injection port 720 after separating the cap 722 when the drug injection port 720 is used.

The connection cap 730 has a body 732 and protrusions 734.

A first end of the connection cap 730 is formed in a cylindrical shape surrounding the outer side of the supply pipe 705 and is coupled to the supply pipe 705 to be able to rotate about the extension direction of the supply pipe 705, and a second end thereof expands rearward from the first end while maintaining the cylindrical shape.

In detail, a first end of the body 732 is formed in a cylindrical shape surrounding the guide 708 and has a ring-shaped groove therein in which the outer side of the guide 708 is partially inserted, whereby the body 732 is coupled to be able rotate with the guide 708.

A second end 732 expands rearward while maintaining the cylindrical shape, the inner surface of the body 732 extends rearward to be gradually spaced apart from the supply body 706, and a thread is formed on the inner surface of the second end.

A space in which the second end of the drug solution injection pipe 702 is formed between the inner surface of the second end of the body 732 and the inner surface of the second end of the supply body 706. When a plurality of stopcocks 140 is disposed front and rear, the second ends of the drug solution injection pipes 702 can be thread-fastened to the inner surfaces of the second end of the bodies 732 at the outside of the second end of the supply body 706 y rotating the bodies 732.

First ends of the protrusions 134 are spaced predetermined distances apart from each other in the rotation direction of the body 732 and protrude in bar shapes on the outer surface of first end of the body 732, and second ends thereof extend a predetermined distance toward the second end of the body 732 along the outer surface of the body 732.

Accordingly, a user can hold the protrusions 734 with fingers and can conveniently rotate the body 732. Further, since the second end of the body 732 thread-fastened to the second end of the drug solution injection pipe 702 expands, separation of the drug solution injection pipe 702 is effectively prevented.

The stopper 140 is formed in a horseshoe shape protruding rearward and the protruding outer surface is coupled to the center part 701 in close contact with the inner surface of the center part 701 at a position corresponding to the lower portion of the cap 712.

The stopper 710 supports the cap 712 by being fitted on the inner surface of the center part 701 and moves toward the lower portion of the cap 712 due to deformation of the blocking diaphragm 714, thereby preventing the blocking diaphragm 714 from moving toward the lower portion of the passage 20.

The ring 750 is formed in a hollow circular frame shape and is fitted on the inner surface of the center part 701 at a position corresponding to the upper portion of the cap 712, thereby preventing separation of the cap 712 from the center part 701.

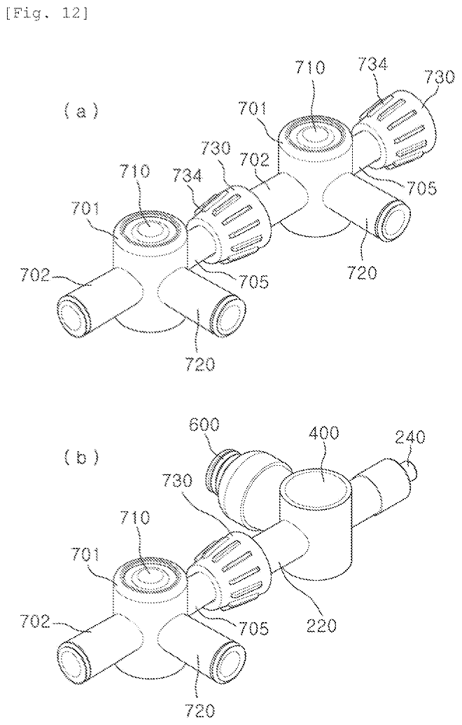

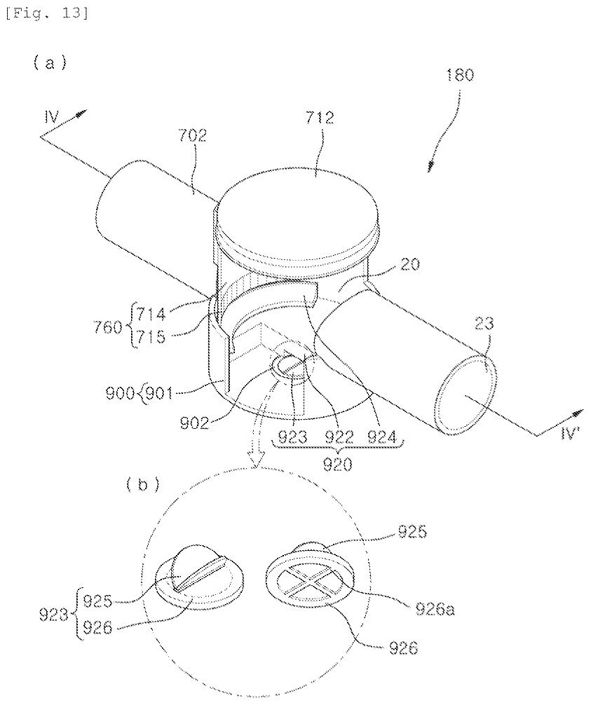

FIG. 11 is a cross-sectional view showing a stopcock according to another embodiment of the present disclosure. FIG. 12 is a perspective view showing an example of another state of the stopcocks shown in FIGS. 1 and 11.

Since a stopcock 160 according to this embodiment is substantially the same as the stopcock 140 shown in FIGS. 7 to 10 except for a shock absorber 800, the same reference numerals and terms are used and repeated description is omitted.

The stopcock 160 further includes a shock absorber 800.

The shock absorber 800 is disposed in the stopcock 160, thereby preventing a needle inserted into the passage 20 through the cap 712 of the backflow prevention unit 710 from being damaged by hitting against the lower inner surface of the center part 701.

In detail, the shock absorber 800 is formed in a cylindrical shape and disposed in the lower portion of the passage 20 and the outer surface of the shock absorber 800 is in close contact with the inner surface of the center part 701 in the passage 20.

The bottom of the shock absorber 800 is in close contact the inner surface of the center part 701 which corresponds to the lower portion of the passage 20, and the top of the shock absorber 800 is positioned lower than the space in which the passages 20 and 22 are connected to each other and is spaced a predetermined distance apart from the second end of the blocking diaphragm 711.

The shock absorber 800 is made of an elastic polymeric material or a natural material lower in strength than the needle, may be made of porous material having fine holes, and may be made of an innoxious silicon material.

Accordingly, when the needle of a syringe is excessively inserted into the passage 20 through the cap 112, the needle is inserted into the shock absorber 800 disposed opposite to the cap 712 with the passage 20 therebetween, whereby damage to the needle is prevented.

Referring to FIG. 13, a plurality of stopcocks 160 can be connected to each other by thread-fastening the connection caps 730 and the drug solution injection pipes 702, and may also be thread-fastened between the stopcock 100 and the stopcock 160 shown in FIGS. 1 to 6.

In detail, when the connection cap 730 is rotated forward with the second end of the drug solution injection pipe 702 partially inserted in the space formed between the second end of the connection cap 730 and the second end of the supply pipe 105, the inner surface of the connection cap 730 and the outer surface of the drug solution injection pipe 702 are thread-fastened so each other.

When it is required to separate a plurality of stopcocks 160 combined with each other, a user can separate the connection cap 730 and the drug solution injection pipe 702 from each other by holding the protrusions 734 with fingers and rotating the connection cap 730 backward.

Referring to FIG. 12(b), the stopcocks 100 and 160 may be detachably coupled to each other through the connection cap 740 and the drug solution injection pipe 220. Further, it is possible to inject various drugs into the stopcocks 100 and 160 through the drug solution injection pipe 702, the drug injection ports 720 and 600, and the backflow prevention units 710 and 400 with the stopcocks 100 and 160 combined with each other.

In detail, a thread that can be engaged with the thread formed on the inner side of the second end of the connection cap 730 may be formed on the outer side of the second end of the drug solution injection pipe 220, and the second end of the drug solution injection pipe 220 can be inserted between the connection cap 730 and the supply pipe 705.

With the second end of the drug solution injection pipe 220 inserted between the connection cap 730 and the supply pipe 705, when a user rotates forward the connection cap 730 with fingers, the drug solution injection pipe 220 and the connection cap 730 are thread-fastened to each other.

On the other hand, with the stopcocks 100 and 160 thread-fastened to each other, when a user rotates backward the connection cap 730, the drug solution injection pipe 220 and the connection cap 730 are separated from each other.

Accordingly, a user can connect a plurality of stopcocks 100, 140, and 160 and inject various drugs into a human body through the stopcocks 100, 140, and 160, depending on environments.

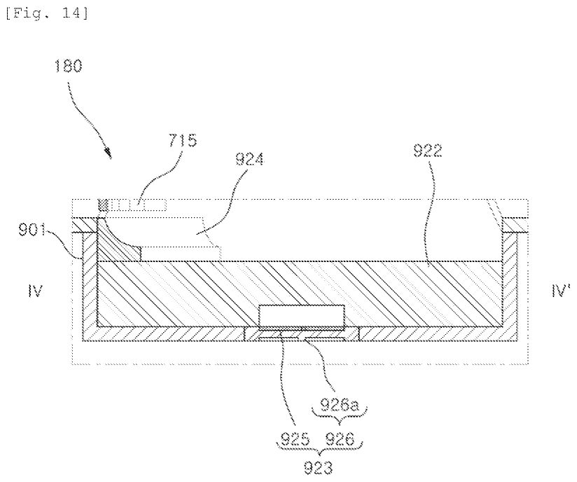

FIG. 13 is a perspective view showing a stopcock according to another embodiment of the present disclosure. FIG. 14 is a cross-sectional view of the stopcock shown in FIG. 13 taken along line IV-IV′.

Since a stopcock 180 according to this embodiment is substantially the same as the stopcock 140 shown in FIGS. 7 to 10 except for a backflow prevention unit 760, a body 900, and a shock absorber 920, the same reference numerals and terms are used and repeated description is omitted.

Referring to FIGS. 13 and 14, the body 900 has a center part 901.

The body 900 is elongated forward or rearward, has passages elongated in the extension direction therein, and has a center portion having a cylindrical shape extending perpendicular to the extension direction.

The center part 901 is formed in a cylindrical shape having a passage 20 therein that is open upward, has a hole 902 formed through the bottom thereof and connecting the passage to the outside, and connects the passages 22 and 23 of the drug solution injection pipe 720 and the supply pipe 705 formed at the front and rear, respectively, to each other through the passage 20.

The shock absorber 920 includes a shock-absorbing body 922, a door 923, and a magnetic part 924.

The shock absorber 920 is cylindrically formed and disposed in the lower portion of the passage 20, has a bottom being in close contact with the bottom inside the center part 901 which corresponds to the lower portion of the passage 20, and has an outer surface being in close contact with the outer surface of the center part 901.

The shock-absorber 920 is inserted in the center part 901 to be able to rotate about a shaft extending upward or downward at the center and the center of the lower end of the shock absorber 920 is partially inserted in the hole 902, whereby enabling a needle inserted through the hole 902 from under the center part 901 to be moved toward the passage 20 and preventing a needle inserted into the passage 20 through she cap 712 from moving to the outside.

The edge of the upper end of the shock absorber 920 is partially made of a magnetic material and it is possible to control movement of a drug between the drug solution injection pipe 702 and the center part 901 by controlling the degree of deformation of blocking diaphragm 714 and an elastic diaphragm 115 to be described below in accordance with the rotation distance of the shock absorber 920.

In detail, the shock-absorbing body 922 is cylindrically formed and inserted in the lower portion of the passage 20, has a bottom being in close contact with the bottom inside the center part 901 which is positioned at the lower portion of the passage 20, and has an outer surface being in close contact with the inner surface of the center part 901 which is positioned on a side of the passage 20.

The center of the bottom of the shock-absorbing body 922 is partially recessed a predetermined distance upward in a cylindrical shape over the hole 902, whereby a lower groove is formed. The diameter of the cross-section of the lower groove is smaller than the diameter of the hollow 902.

The door 923 is formed in a circular plate shape and inserted in the hole 902, the outer surface thereof is in close contact with the inner surface of the center part 901 which is positioned outside the hole 902, and the edge of the top thereof is partially coupled to the bottom of the shock-absorbing body 922.

A film of which both sides can be part ally deformed is attached to the upper end of the door 923, so when a needle is inserted through the hole 902, the film is deformed, thereby providing a passage through which the needle can be moved to the passage 20.

On the other hand, when a needle inserted into the passage 20 through the cap 712 is moved to the door through the shock-absorbing body 922, the film of the door 923 prevents damage to the needle while stopping the needle.

In detail, the door 923 includes a film 925 and a through-plate 926.

The through-plate 926, which is a circular plate, is inserted in the hollow 902 and the outer surface thereof is disposed in the hole 902 to be able to rotate about an axis extending upward or downward at the center in close contact with the center part 901.

A portion of the edge of the top of the through-plate 926 partially extends a predetermined distance upward and is coupled to the bottom of the shock-absorbing body 922, so when the through-plate 926 is rotated, the shock-absorbing body 922 is rotated in the same direction as the rotation direction of the through-plate 926 in the passage 20.

A circular plate of the center of the bottom of the through-plate 926 is partially recessed a predetermined distance upward, whereby a center groove is formed. The thickness of the through-plate at the center groove is smaller than the thickness of a portion of the edge of the through-plate 926.

A protrusion 926a protruding a predetermined distance downward from the bottom of the through-plate 926 is formed in a cross shape inside the center groove and is used as a handle for rotating the through plate 926. Fan-shaped grooves formed between the bottom edges of the protrusion 926a and the through-plate 926 are used as spaces for guiding a needle that is inserted.

The film 925 is formed in a circular shape or a plate shape and is coupled to the top of the through-plate 926 through a bar-shaped center portion extending toward a side or another side across the center, and the front and rear portions of the film 925 is deformed upward and separated from the through-plate 926 with the center portion therebetween.

Accordingly, a needle passing through the through-plate 926 through a space formed between the bottom edges of the protrusion 926a and the through-plate 926 can pass through the shock-absorbing body 922 while pushing up the bottom of the film 925, whereby a drug solution can be injected into the passage 20.

When a needle inserted into the passage 20 through the cap 712 hits against the door 923 through the shock-absorbing body 922 by excessively descending, the film 925 stops the needle and the shock that is applied to the film 925 is attenuated by deformation of the shock-absorbing body 922 connected to the through-plate 926. Accordingly, there is an advantage that damage to a needle is prevented.

A first end of the magnetic part 924 is coupled to the edge of the top of the shock-absorbing body 922 at a position corresponding to the lower portion of the space in which the passages 20 and 22 are connected to each other, and a second end thereof extends a predetermined distance in an arc shape along the edge of the shock-absorbing body 922.

The edge of the upper end of the inner side of the magnetic part 924 is recessed a predetermined distance toward the edge of the top of the shock-absorbing body 922, whereby a curved surface protruding toward the edge of the top of the shock-absorbing body 922 is formed. Accordingly, the curved surface functions as a guide surface enabling a drug flowing through the passage 22 to smoothly move to the passage 20.

The magnetic part. 924 is made of a rubber magnet or a metal magnet having magnetism, the distance from an elastic diaphragm. 715 to be described below is changed in accordance with the rotation angle of the shock-absorbing body 922 through the protrusion 926a, and elasticity of the elastic diaphragm 715 and the blocking diaphragm 714 can be controlled by the change of the distance from the elastic diaphragm 115.

The backflow prevention unit 160 further includes the elastic diaphragm 715.

A first end of the backflow prevention unit 760 is coupled to a side of the body 900 between the drug solution injection pipe 702 and the supply pipe 705, and a second end thereof extends into the body 900 to isolate the drug solution injection pipe 702 and the body 900 from each other.

A first end of the elastic diaphragm 715 is connected to the second end of the blocking diaphragm. 714 in the same shape as the blocking diaphragm 114 and a second end thereof extends a predetermined distance downward from the first end to be close to the magnetic part 924.

The elastic diaphragm 715 is a magnetic body having a different magnetic pole from the magnetic part 924 and has a characteristic of moving toward the magnetic part 921 by magnetism at a position close to the magnetic part 924.

Accordingly, when the shock absorber 920 is rotated and the magnetic part 924 reaches a position close to the elastic diaphragm 715, the shape-maintaining ability of the blocking diaphragm 714 increases and deformation of the blocking diaphragm 714 is suppressed. When the magnetic part 924 is moved away from the elastic diaphragm 715, the blocking diaphragm 714 is relatively easily deformed.

Further, when a drug solution injected into the passage 20 from the drug solution injection pipe 702 has difficulty in moving due to a defect of the blocking diaphragm 714 or the blocking diaphragm. 714 cannot isolate the passages 20 and 22, a user can cope with the defect of the blocking diaphragm 714 because it is possible to control deformation of the blocking diaphragm 714 by rotating the shock absorber 920.

Although embodiments of the present disclosure were described above with reference to the accompanying drawings, the configurations described in the embodiments and drawings of the present disclosure are merely most preferable embodiments but do not represent all of the technical spirit of the present disclosure. Thus, the present disclosure should be construed as including all the changes, equivalents, and substitutions included in the spirit and scope of the present disclosure at the time of filing this application. Therefore, it should be understood that the embodiment described above is not limitative, but only an example in all respects, the scope of the present disclosure, is expressed by claims described below, not the detailed description, and it should be construed that all of changes and modifications achieved from the meanings and scope of claims and equivalent concept are included in the scope of the present disclosure.

### INDUSTRIAL APPLICABILITY

A stopcock, which is a device configured to be connected with transfusion tubes and controlling movement of various drug solutions, can be used for home use or medical use.

1. A stopcock comprising:
a body formed in a passage shape connecting a drug solution injection pipe and a supply pipe to each other; and
a backflow prevention unit having a first end coupled to a side of the body between the drug solution injection pipe and the supply pipe and a second end extending toward an inside of the body to isolate the drug solution injection pipe and the supply pipe from each other,
wherein the second end of the backflow prevention unit bends toward the inside of the body and guides movement of a drug when the drug is injected through the drug solution injection pipe, and elastically moves to an initial position when injection of the drug is stopped.

2. The stopcock of claim 1, wherein the first end of the drug solution injection pipe is formed in a cylinder shape, the second end of the drug solution injection pipe is formed by extending in a plate shape from a portion of an edge of the first end in close contact with an inner surface of the body, and a groove recessed toward the drug solution injection pipe is formed at the second end.

3. The stopcock of claim 1, wherein the body further comprises a drug injection pipe coupled to an outer side of the body at a position spaced apart from the backflow prevention unit between the drug solution injection pipe and the supply pipe, and having a second end extending outward in a tube shape and connecting the inside of the body to the outside.

4. The stopcock of claim 3, further comprising a drug injection port having a first end formed in a tube shape and detachably coupled to the second end of the drug injection pipe, and a second end extending away from the body to seal the drug injection pipe.

5. The stopcock of claim 4, wherein the drug injection port comprises: a housing having a first end formed in a tube shape and surrounding the drug injection pipe and a second end extending and tapering away from the body; and a cap having a first end inserted in the second end of the housing, a second end expanding and extending toward the body from an edge of the first end in close contact with an inner surface of the drug injection pipe, and a hole formed through a side thereof.

6. The stopcock of claim 1, wherein the backflow prevention unit has a first end having a ring-shaped protrusion surrounding an outer side thereof, and inserted in a side of the body, and a second end extending into the body in close contact with an inner surface of the body from a portion of an edge of the first end.

7. The stopcock of claim 6, further comprising a shock absorber formed inside another side of the body to be spaced apart from the backflow prevention unit with a predetermined space formed in the body therebetween.

8. The stopcock of claim 1, further comprising a connection cap having a first end coupled to be able to rotate with the supply pipe, a second end expanding and extending from the first end, and a thread formed inside the second end to be engaged with an outer side of the drug solution injection pipe.
